(19) United States
(12) Patent Application Publication
(10) Pub. No.: US 2004/0033492 A1
(43) Pub. Date: Feb. 19, 2004

(54) Nucleic acid sequencing method

(76) Inventor: Chi-Ming Chen, Taipei (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(21) Appl. No.: 10/065,610

(22) Filed: Nov. 4, 2002

(30) Foreign Application Priority Data
Aug. 16, 2002 (TW) 91118507

(51) Int. Cl. C12Q 1/68

(52) U.S. Cl. 435/6; 205/777.5

(57) ABSTRACT

The present invention relates to a nucleic acid sequencing method by using a rotating electric field to control translocation of a polynucleotide sequence through a nanopore. The translocation time of each nucleotide passing through the nanopore is found to be a multiple of ¼ the period of the rotating electric field. By comparing change of the blockage current for the polynucleotide sequence through the nanopore over time and measuring the translocation time, the linking order of the nucleotides in the polynucleotide sequence and the repeating nucleotide numbers can be determined. Therefore, a rapid nucleic acid sequencing method is provided when the rotating electric filed is adjusted to an adequate frequency.

### CROSS REFERENCE TO RELATED APPLICATIONS

This application claims the priority benefit of Taiwan application serial no. 91118507, filed Aug. 16, 2002.

### BACKGROUND OF INVENTION

1. Field of Invention

The present invention relates to a nucleic acid sequencing method. More particularly, the present invention relates to a nucleic acid sequencing method for rapid sequencing by a rotating electric field. In the present invention, the nucleic acid includes deoxyribonucleic acid (DNA) and ribonucleic acid (RNA).

2. Description of Related Art

After the human genome project, it has become promising to use gene sequencing to diagnose or treat genetic diseases. Therefore, much research has been initiated to develop the methods and/or instrumentation for gene or nucleic acid sequencing.

The prior art nucleic acid sequencing method proposed by Fredrick Sanger is performed by replicating DNA under controlled conditions to obtain fragments of various lengths, so that the complete DNA sequence can be derived. The following paragraph details the prior art nucleic acid sequencing method.

Several polymerase chain reaction (PCR) reagents, including polymerase I, specific primers with complementary sequences, deoxyribonucleotide triphosphate (dNTPs) and buffers, are provided. The dNTPs are labeled with either isotopes or fluorescent molecules. On the other hand, dNTP analogs are prepared. The dNTP analogs lack the 3″-hydroxyl groups for forming the phosphodiester bond with the next subunit, thus terminating the elongation of DNA. After four different dNTP analogs are added into four groups of PCR reagents respectively, four groups of chain terminated fragments finished with four dNTP analogs are obtained through PCR reactions. Later on, electrophoresis is used to separate DNA fragments with diversified and different lengths. The DNA fragments are detected either through isotopes or fluorescent molecules. By comparing the sizes (or the positions and spacing in the gel) of each fragment, the DNA sequence is obtained.

Although DNA sequencing has important medical applications, so far the prior art methods in sequencing nucleic acid (or polynucleotides) are time-consuming, costly, and inaccurate. For example, the prior Sanger method used for human genome sequencing took 15 years and cost nearly 3 billion USD. Not only are the PCR reaction and electrophoresis analysis very time-consuming, but the instrumentation and reagents are also very expensive. Because of its slowness and high-price, the prior art DNA sequencing technique is impractical in diagnosing diseases, especially acute or epidemic diseases.

Moreover, accuracy problems exist in the prior art DNA sequencing method, as shown in Cell, 106, 413 (2001). A comparison of Celera and Ensembl predicted gene sets reveals 20% overlap in novel genes. It is expected that the similarity of uncoded regions between these two groups” results is even smaller. Therefore, it is highly desirable to develop an accurate nucleic acid sequencing method that is fast and inexpensive.

### SUMMARY OF INVENTION

The invention provides a nucleic acid sequencing method, which is time-efficient and low-cost.

The invention provides a fast single-molecule nucleic acid sequencing method with higher accuracy.

As embodied and broadly described herein, the present invention provides a nucleic acid sequencing method. After providing a thin film with a nanopore and placing the thin film in a buffer solution, nucleic acid sequences are added into the buffer solution. The nucleic acid sequence can be a DNA sequence or an RNA sequence. An applied electric field perpendicular to the thin film drives the nucleic acid sequence to pass through the nanopore of the thin film. At the same time, a rotating electric field parallel to the thin film is applied to control the movement (i.e. the translocation speed) of the nucleic acid sequence through the nanopore. The rotating electric field controls whether the nucleic acid sequence is stretched or unstretched. The frequency of the rotating electric field correlates to the translocation speed of the nucleic acid sequence through the nanopore. For a rotating electric field with high frequency, the polynucleotide sequence will rapidly pass through the nanopore. On the other hand, for a rotating electric field with low(er) frequency, the translocation speed of the polynucleotide sequence is under control. With an adequate frequency, the rotating electric field can control only one nucleotide of the polynucleotide sequence passing through the nanopore at a time. Since different nucleotides (i.e. A, G, T, C) cause different levels of blockage toward the nanopore, measured blockage currents for different kind of nucleotides are distinct. In the present invention, an outer circuit is applied to measure the blockage currents and, change of blockage currents over time, so that the polynucleotide sequence can be determined by measuring the change of blockage currents over time. The method of the present invention further includes adding two extra fragments at both ends of the tested sequence respectively. These two fragments can be used to label different ends (3″ or 5″ end) and to locate the main sequence.

The sequencing method of the present invention can be performed in the array format by forming array cells in the thin film and forming one nanopore in each cell. Such sequencing array design is useful in making comparison for different sets of results from the same nucleic acid sequence. Sincethe translocation time of each nucleotide is in multiples of T_{c}/4 (time for the sequence to remain stretching) and each kind of nucleotide has a distinct blockage current, numbers of repeating nucleotides in sections of the polynucleotide sequence can be obtained by comparing the measured time of a specific section (in unit of T_{c}/4) to obtain the smallest integer multiple. The change of blockage currents over time of the same nucleic acid sequence is measured several times, in order to obtain different sets of results. By making comparisons between different sets of results from the same sequence, prediction errors can be greatly reduced and the polynucleotide sequence can be determined accurately.

It is to be understood that both the foregoing general description and the following detailed description are exemplary, and are intended to provide further explanation of the invention as claimed.

### BRIEF DESCRIPTION OF DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a display view of a nucleic acid sequence passing a nanopore of a thin film according to one preferred embodiment of the present invention;

FIG. 2 is a display view of a nucleic acid sequence passing a nanopore of a thin film under the influence of a rotating electric field and a stable electric field according to one preferred embodiment of the present invention;

FIG. 3 is a display view showing a simulated polynucleotide sequence passing through the nanopore according to the bond-fluctuation model in a cubic lattice;

FIG. 4 is a display view showing a simulated polynucleotide sequence passing through the nanopore according to the off-lattice bead-spring model;

FIGS. 5A and 5B show the relationship of the numbers of nucleotides that have passed the pore versus time in the translocation processes under high frequencies and low frequencies, respectively;

FIG. 6 shows quantization of the translocation time for each nucleotide passing through the nanopore under low frequency rotating electric field, while the nucleotides are marked according to the lengths of their translocation time, not their order in the sequence;

FIG. 7 shows the relationship of the translocation time for the polynucleotide sequence passing through the pore versus the frequency of the rotating electric field. The inset shows the translocation time of each nucleotide of the polynucleotide during a typical translocation process simulated by the off-lattice bead-spring model;

FIG. 8A shows the relationship of the number of nucleotides passing through the nanopore versus time, while FIG. 8B illustrates the relationship between the translocation time of the nucleotides in FIG. 8A and their measured blockage currents; and

FIG. 9 shows the relationship of the prediction errors versus the number of the measurements.

### DETAILED DESCRIPTION

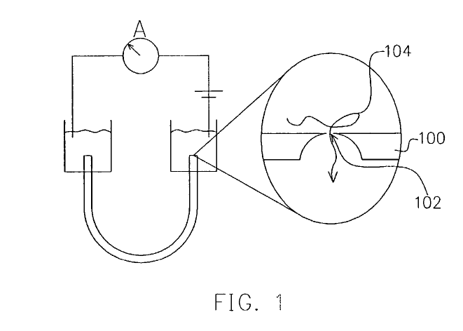

FIG. 1 is a display view of a nucleic acid sequence passing a pore of a thin film according to one preferred embodiment of the present invention.

As shown in FIG. 1, a membrane or thin film 100 is provided with a nanopore 102. The thin film 100 is made of, for example, silicon nitride. For example, an ion beam is used to form the nanopore 102 and the nanopore 102 has a size of about 2-3 nm. For the method for forming nanopores in the thin film refer to J. Li et al., Nature 412, 166 (2001).

After the thin film 100 is placed in a buffer solution 106 (as shown in FIG. 1), nucleic acid sequences 104 are added into the buffer solution 106. The nucleic acid sequence 104 can be a DNA sequence or an RNA sequence, and sometimes is denoted as a polynucleotide sequence or a polynucleotide. Since the nucleic acid sequence 104 is a long chain with negative charges, an applied electric field perpendicular to the thin film can drive the nucleic acid sequence 104 to pass through the nanopore 102 of the thin film 100.

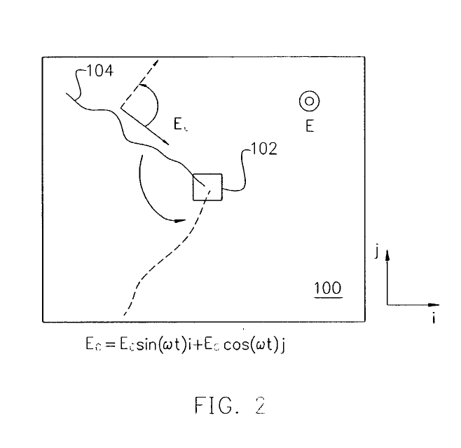

As shown in FIG. 2, the nucleic acid sequence 104 is driven by a uniform-amplitude electric field Ein the z-direction to pass through a nanopore of size D in the thin film 100. A rotating electric field E_{c} on the x-y plane is added in order to control the movement (i.e. the translocation speed) of the nucleic acid sequence 104 through the nanopore 102.

Therefore, except for the applied electric field E perpendicular to the thin film that drives the nucleic acid sequence 104 to pass through the nanopore 102 of the thin film 100, the rotating electric field E_{c} parallel to the thin film 100 controls stretching or unstretching (releasing) the long-chain nucleic acid sequence 104 so as to control the translocation speed of the nucleic acid sequence 104 through the nanopore 102.

According to the preferred embodiment, the rotating electric field E_{c} is formed by one set of parallel electrode pairs perpendicular to another set of parallel electrode pairs. One set of parallel electrode pairs generates a sinusoid (sine) AC electric field, while the other set of parallel electrode pairs generates a cosinusoid (cosine) AC electric field. With the same frequency, the combination of these two electric field having a 90-degree phase difference forms a circular rotating electric field E_{c}=E_{c} sin(ωt)i+E_{c} cos (ωt)j, while i and j are unit vectors in the x- and y-directions, as shown in FIG. 2.

The rotating electric field E_{c} controls whether the nucleic acid sequence 104 is stretched or unstretched. If the nucleic acid sequence 104 is fully stretched by the rotating electric field E_{c}, the nucleic acid sequence 104 above the nanopore 102 cannot travel through the nanopore 102. Only if the nucleic acid sequence 104 becomes relaxed (unstretched) by the rotating electric field E_{c}, can the nucleic acid sequence 104 above the nanopore 102 travel through the nanopore 102. The frequency ω of the rotating electric field E_{c} correlates to the translocation speed of the nucleic acid sequence 104 through the nanopore 102. For a rotating electric field with high frequency, the polynucleotide sequence rapidly passes through the nanopore. On the other hand, for a rotating electric field with low(er) frequency, the translocation speed of the polynucleotide sequence is controlled. With an adequate frequency, the rotating electric field can be controlled to only one nucleotide of the polynucleotide sequence passing through the nanopore 102 at a time. Moreover, the translocation time (i.e. the time required for passing through the nanopore) of each nucleotide is found to be mT_{c}/4, where m is an integer and T_{c}/4 is the time that the sequence remains stretching, while T_{c} is the period of the rotating electric field.

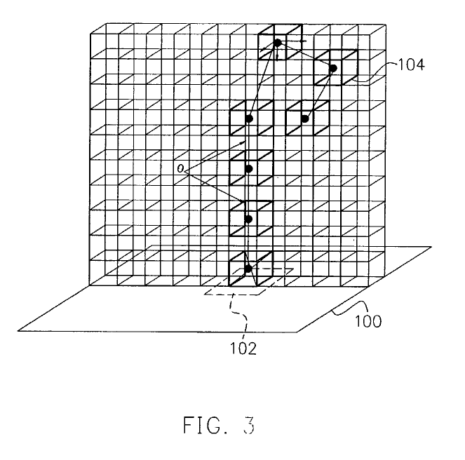

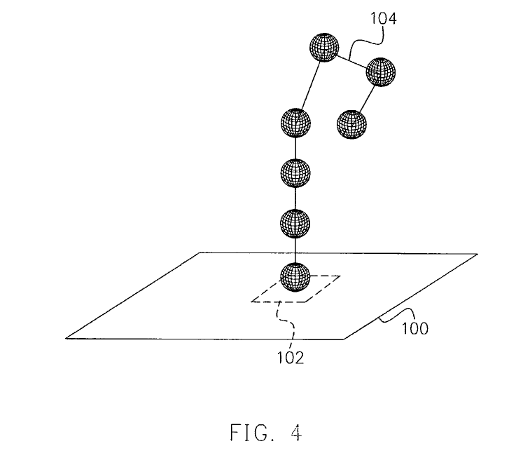

FIGS. 3 and 4 are display views showing a simulated polynucleotide sequence passing through the nanopore.

Referring to FIGS. 3 and 4, a polynucleotide sequence 104 of length N (i.e. having N nucleotides) is represented by the bond-fluctuation model in a cubic lattice (as shown in FIG. 3) or the off-lattice bead-spring model (as shown in Fig. 4). In the preferred embodiment, the polynucleotide sequence 104 is a single strand DNA (ssDNA), and the Metropolis Monte-Carlo (MC) algorithm at a constant temperature T is used to simulate its motion.

In the bond-fluctuation model, each nucleotide occupies a cube of length 1 (lattice spacing) and the set of allowed bond vectors is B=P(2,0,0)νP(2,1,0)νP (2,1,1)νP(2,2,1)νP(3,0,0)νP(3,1,0), where P(a, b, c) stands for the set of all permutations and sign combinations of ±a,±b,±c. This model has been shown to be a realistic and efficient method for studying polymer dynamics in various systems and has been cited in a few references, including C.-M. Chen, Y.-A. Fwu, Phys. Rev. E63, 011506(2001).

Referring to FIGS. 2-4, in the simulations, the nucleic acid sequence (polynucleotide sequence) 104 is driven by the uniform electric field E in the z-direction (perpendicular to the thin film 100) to pass through the nanopore 102 of the thin film 100. Above the thin film, the electric field E_{c} rotates on the x-y plane (parallel to the thin film 100) to control the translocation speed of the polynucleotide 104 passing the nanopore 102. Both the frequency and the amplitude of the rotating field can be used to control the movement (i.e. translocation speed) for each nucleotide of the polynucleotide sequence.

At each instant, a nucleotide is picked up at random and attempts to move in any of the six directions by one lattice spacing. If any attempted move of nucleotides satisfies the excluded volume constraint and the new bond vectors are still in the allowed set, the move is accepted with probability p=min[1,exp(−66 U/kT)], where Δ U is the energy change of the chain and kT is thermal energy. In this model, the energy of polynucleotide sequence 104 is expressed as U=U_{bend}+U_{electric}+U_{H-bond}, where U_{bend}=Σᵢe(1 cos θ_{ι}) is the bending energy of the chain with a rigidity e and a bending angle θ_{ι}·U_{electric} is the electric potential energy due to a constant electric field in the z-direction and a rotating electric field on the x-y plane, and U_{H-bond} is the hydrogen bonding energy of (A, T) and (G, C) pairs. Here it is considered to have negligible hydrogen bonding between bases, which can be realized by adjusting pH value, raising temperature, or adding urea.

To study the kinetics of the polynucleotide 104 passing through the nanopore 102, each set of parameters for the translocation process is simulated 50 times. For a chain of 50 nucleotides, we choose the pore size D=3, temperature T=1, the uniform electric field amplitude E=1.5, and the bending rigidity e=0.2. Here thermal energy and electric charge of each nucleotide are set to unity, and the corresponding electric field is of order 10⁷ V/m. For the rotating field, its frequency ω varies from 10⁻¹ to 10⁻⁸ (MC step⁻¹) and its amplitude E_{c} varies from 0.1 to 1.2.

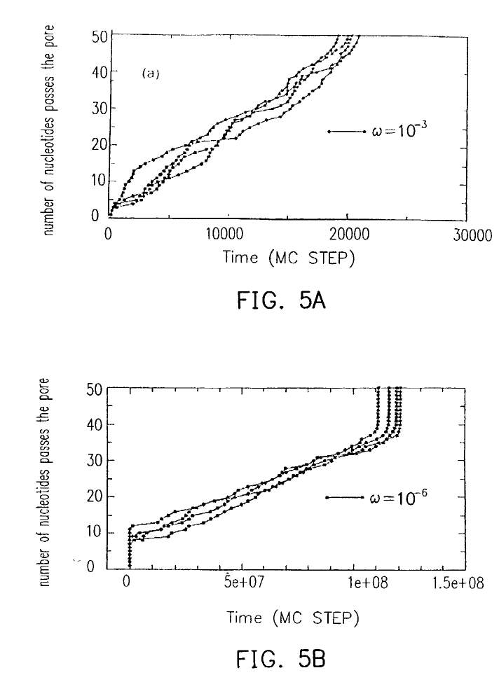

FIGS. 5A and 5B show the relationships of the numbers of nucleotides that have passed the pore versus time in the translocation process under high frequencies and low frequencies, respectively. As shown in FIG. 5A, at high frequencies (ω≧10⁻³), the polynucleotide sequence passes through the pore smoothly and the translocation time of the whole chain, t_{c}, is about a constant (t_{c}˜2×10⁴ MC steps). In these cases, the translocation time of each nucleotide, tₙ, does not vary dramatically.

As shown in FIG. 5B, at low frequencies (ω≦10⁻⁴), two kinds of translocation kinetics are observed for the polynucleotide sequence. For nucleotides located at the middle of the sequence, tₙ is much longer than that of nucleotides near both ends of the sequence. At ω=10⁻⁶, t_{c} is about 10⁸ MC steps. It has been estimated previously that tₙ is about 1 microsecond at the present driving electric field strength for a smooth translocation and thus 1 MC step in the simulation is in the order of 10⁻⁸ sec. It is concluded that the frequency of the rotating field should be less than 10⁴ Hz in order to slow down the translocation process.

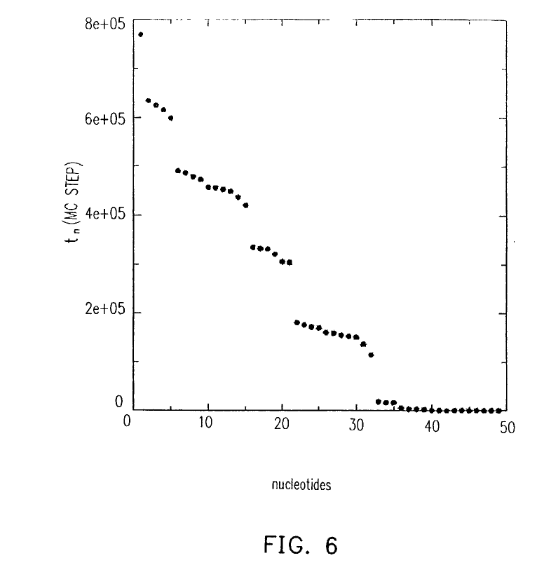

FIG. 6 shows quantization of the translocation time for each nucleotide passing through the nanopore under a low frequency rotating electric field, while the nucleotides are marked according to the lengths of their translocation time, but not their order in the sequence.

As shown in FIG. 6, the two axes are nucleotide versus the translocation time tₙ each nucleotide. The detailed study reveals that tₙ≈mT_{c}/4 for ω≦10⁻⁴ and E_{c}/E>0.4, where m is an integer and T_{c}=2 π/ω. This effect of quantized tₙ can be explained as follows. At some instant, the remaining segment of the chain (the polynucleotide sequence) above the thin film is aligned along the direction of the rotating electric field. In this case, the chain is taut and the nucleotide near the nanopore cannot pass through the pore. The stretched polynucleotide chain does not move with the rotating field due to lattice effects when the rotating field rotates away from the aligned direction. When the rotating field is almost perpendicular to the aligned direction, the stretched chain starts to move and becomes loose (unstretched). If the response of the chain is faster than the rotation of the rotating electric field, it will be quickly aligned along the new direction of the rotating field again. Since the nucleotide near the pore can pass through the pore only when the chain is loose, tₙ must be a multiple of T_{c}/4. Deviation of tₙ from predicted values would depend on the stability of the stretched chain and its response toward the rotating electric field.

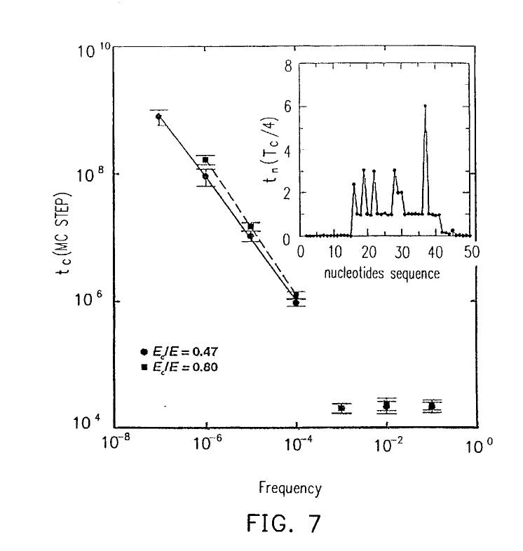

FIG. 7 shows the translocation time for the polynucleotide sequence passing through the pore versus the frequency of the rotating electric field. In order to eliminate possible lattice effects, off-lattice simulations of the polynucleotide translocation process are also carried out. The inset of FIG. 7 shows the translocation time of each nucleotide of the polynucleotide in a typical translocation process using the off-lattice bead-spring model, in which the quantization of tₙ is clear. The simulated polynucleotide sequence in the off lattice bead-spring model behaves differently from that in the cubic lattice bond-fluctuation model. The polynucleotide sequence in the off lattice model cannot pass through the thin film as a whole under low frequency rotating electric field. However, by transitorily shutting off the rotating electric field or tuning the rotating electric field to a higher frequency (the shutting time is 0.02 period for every ¼ period of the rotating electric field in the inset), the polynucleotide sequence in the off lattice bead-spring model can behave in the same way as the sequence in the cubic lattice bond-fluctuation model, as shown in the inset. Referring to FIG. 7, the two axes are the translocation time t_{c} of the polynucleotide sequence versus the frequency ω of the rotating electric field, showing the dependence of t_{c} on ω. If the response of the chain is too slow, it will always be loose and penetrate the pore smoothly. FIG. 7 shows that t_{c} is inversely proportional to ω for ω≦10⁻⁴ and is almost a constant for ω≧10⁻³. The boundary between these two regimes depends on the response of the polynucleotide sequence and can be varied by changing the viscosity of the solution or the friction of the thin film surface.

The aforementioned circumstances apply to the polynucleotide sequence of 30 nucleotides, 70 nucleotides and 100 nucleotides.

The present invention further includes linking two specific sequence fragments to both ends (the 3″ end and 5″ end) of the polynucleotide sequence, in order to tell the differences between both ends. In the preferred embodiment, we select a polynucleotide consisting of 26 randomly generated nucleotides (GTACTTCGCGTGTAGTCATTTAATCC) located at the middle and two extra fragments AAAAAAAAAAAC and ACCCCCCCCCCC attached at the 3″ and 5″ ends, respectively. These two fragments are added because nucleotides near both ends pass through the nanopore quickly and cannot be sequenced, as indicated in FIG. 5B. In addition, they can be used to locate the main sequence.

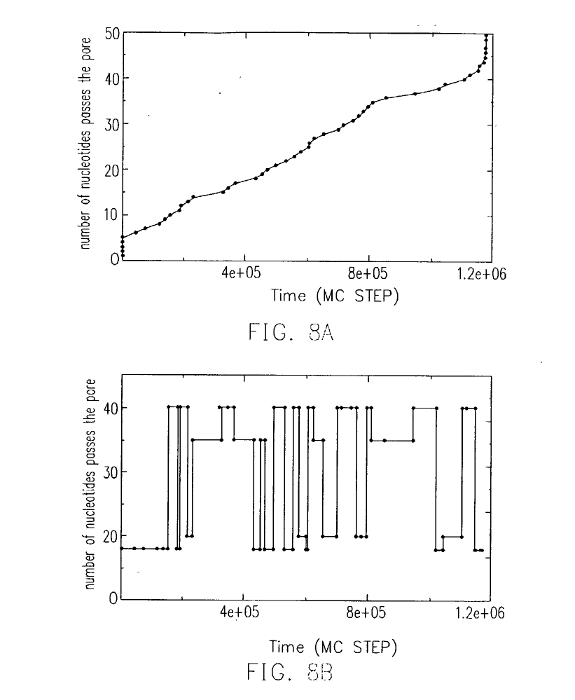

In FIG. 8A, the relationship of the number of nucleotides passing through the nanopore versus time is shown, while FIG. 8B illustrates the relationship between the translocation time of the nucleotides in FIG. 8A and their measured blockage currents. Since different nucleotides (i.e. A, G, T, C) cause different levels of blockage toward the nanopore, the measured blockage currents for different kind of nucleotides are distinct. In the present invention, an outer circuit is applied to measure the blockage currents and change of blockage currents over time, so that the polynucleotide sequence (i.e. the linking order of the nucleotides) can be determined by measuring the change of blockage currents over time. Using the aforementioned 26-nucleotide polynucleotide sequence having two extra fragments AAAAAAAAAAAC and ACCCCCCCCCCC attached at the 3″ and 5″ ends as an example, if the blockage currents of A, G, T, C are assumed to be 18, 20, 35, and 40, an increase in the blockage current from 18 to 40 signals the beginning of the main sequence from the 3″ end, while a drop of the blockage current signals the beginning of the sequence from the 5″ end.

The sequencing method of the present invention can be performed in the array format by forming array cells in the thin film and forming one nanopore in each cell using an ion beam, for example. The stable electric field and the rotating electric field are applied to control translocation of the polynucleotide sequence through the pore. Each of the sequencing array cells can be controlled and measured independently or in rows by applied circuits.

Such sequencing array design is useful in making comparison for different sets of results from the same nucleic acid sequence. Since tₙ (translocation time of each nucleotide) is a multiple of T_{c}/4 (time for the sequence to remain stretching) and each kind of nucleotide has a distinct blockage current, the numbers of repeating nucleotides in sections of the polynucleotide sequence can be obtained by comparing the measured time of a specific section (in unit of T_{c}/4) to obtain the smallest integer multiple. The change of blockage currents over time of the same nucleic acid sequence is measured several times, in order to obtain different sets of results. By making comparisons between different sets of results from the same sequence, prediction errors can be greatly reduced and the polynucleotide sequence can be determined accurately.

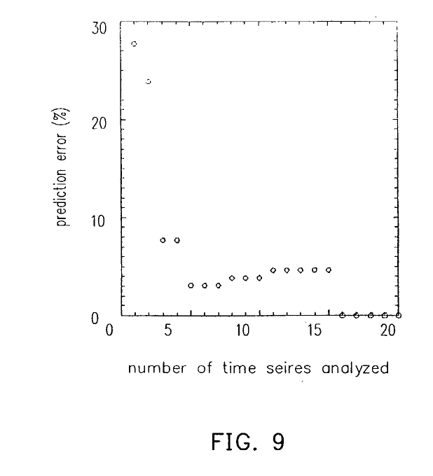

As shown in FIG. 9, the two axes are the prediction errors and the number of the measurements (i.e. being measured for how many times). For one single measurement, the prediction error of the random sequence is about 30%. If more than 16 sets of results are used in analysis and comparison, the sequencing error is reduced to nearly zero. It is evident that the prediction error decreases rapidly as the number of measurements increases.

Note that, experimentally, accuracy of sequencing mainly relies on the differences of blockage currents for (A, G) or (C, T). Adding a chemical group to the specific base of the nucleotide can magnify the differences of blockage currents between different nucleotides. For example, a benzoyl group can be attached to the amino functional groups of A, G, and C.

In the simulations, a strong electric field is considered to be able to reduce thermal effects (for example, backward motion of nucleotides against the driving electric field), and these thermal effects become more significant if a weak electric field is applied. Nevertheless, prediction errors due to thermal effects can be reduced or even cancelled if many sets of measurement results are used for analysis.

In conclusion, the present invention has the following advantages:

1.The present invention provides a rapid single-molecule nucleic acid sequencing method. If performed in the array format, the method of the present invention can determine up to 100 million bases per day.

2.No other reagents or special enzymes are required for the method of the present invention, thus reducing the costs.

3.The present invention provides an accurate nucleic acid sequencing method, in combination of the sequencing array cells. The sequencing error is reduced to nearly zero by analyzing and comparing numerous sets of results obtained from the array cells.

4.According to the method of the present invention, a convenient, accurate and cheap sequencing system can be designed. Such a system can be used to diagnose diseases, in medical treatment or biological sample detection.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A nucleic acid sequencing method, comprising:
providing a thin film having a pore;
disposing at least a nucleic acid sequence on the thin film, wherein the nucleic acid sequence comprises a plurality of nucleotides;
applying an electric field perpendicular to the thin film, so that the nucleic acid sequence passes through the pore, wherein an adjustable rotating electric field parallel to the thin film is applied simultaneously, in order to control a translocation time of one nucleotide being a multiple of one-fourth of a period of the rotating electric field; and
measuring the translocation time of each nucleotide to determine a sequence of the nucleic acid sequence.

2. The method of claim 1, wherein the pore has a size of about 2 to 3 nm.

3. The method of claim 1, wherein the thin film comprises a silicon nitride thin film.

4. The method of claim 1, wherein the pore of the thin film is formed by an ion beam.

5. The method of claim 1, wherein the rotating electric field is formed by one set of parallel electrode pairs perpendicular to another set of parallel electrode pairs, while one set of parallel electrode pairs generate a sinusoid (sine) AC electric field and the other set of parallel electrode pairs generate a cosinusoid (cosine) AC electric field.

6. The method of claim 1, wherein the period of the rotating electric field is smaller than 10⁴ Hz.

7. The method of claim 1, further comprising measuring a blockage current of each nucleotide and analyzing change of the blockage current over time to determine the sequence of the nucleic acid sequence.

8. The method of claim 1, further comprising adding two extra sequence fragments to both ends of the nucleic acid sequence for labeling the both ends.

9. A nucleic acid sequencing method, comprising:
providing array cells formed in a thin film, wherein each array cell has a pore;
disposing at least a nucleic acid in the array cell, where in nucleic acid sequence comprises a plurality of nucleotides;
applying an electric field perpendicular to the thin film, so that the nucleic acid sequence pass through the pore, wherein a adjustable rotating electric field parallel to the thin film is simultaneously applied for controlling translocation times of the nucleotides; and
measuring a blockage current of each nucleotide and analyzing change of the blockage current over time to determine a sequence of the nucleic acid sequence.

10. The method of claim 9, wherein the pore has a size of about 2 to 3 nm.

11. The method of claim 9, wherein the thin film comprises a silicon nitride thin film.

12. The method of claim 9, wherein the pore of the thin film is formed by an ion beam.

13. The method of claim 9, wherein the rotating electric field is formed by one set of parallel electrode pairs perpendicular to another set of parallel electrode pairs, while one set of parallel electrode pairs generate a sinusoid (sine) AC electric field and the other set of parallel electrode pairs generate a cosinusoid (cosine) AC electric field.

14. The method of claim 9, wherein the period of the rotating electric field is smaller than 10⁴ Hz.

15. The method of claim 15, further comprising adding two extra sequence fragments to both ends of the nucleic acid sequence for labeling both ends.
